SEQUENCE LISTING (25 sequences)

SEQ ID NO: 1 - DNA, 38 bp, Artificial sequence
  CMV forward primer
tctagaattctttaattaagtcgttacataacttacgg

SEQ ID NO: 2 - DNA, 39 bp, Artificial sequence
  CMV reverse primer
tctagaattctgcccgggctacaattccgcagcttttag

SEQ ID NO: 3 - DNA, 36 bp, Artificial sequence
  BGH forward primer
ttagtgtgcccgggcactcgctgatcagcctcgact

SEQ ID NO: 4 - DNA, 31 bp, Artificial sequence
  BGH reverse primer
tagtgtctcgagaatcctcccccttgctgtc

SEQ ID NO: 5 - DNA, 32 bp, Artificial sequence
  EF1 forward primer
ttagtgtctcgagaactaacatacgctctcca

SEQ ID NO: 6 - DNA, 57 bp, Artificial sequence
  EF1 Reverse primer
gtgtctgcaggtatttaaatgtgggaattcgtcctaggccctcctaccggtgatctc

SEQ ID NO: 7 - DNA, 35 bp, Artificial sequence
  Heavy chain cDNA forward primer
tacttcgcccgggctaattcgccgccaccatggaa

SEQ ID NO: 8 - DNA, 39 bp, Artificial sequence
  Heavy chain cDNA Reverse primer
tacttcgcccgggctttattcatttacccggagacaggg

SEQ ID NO: 9 - DNA, 34 bp, Artificial sequence
  Light Chain forward primer
cctcacctaggccaccatgggtgtgccacgctgg

SEQ ID NO: 10 - DNA, 30 bp, Artificial sequence
  Light chain reverse primer
cctcacctaggattaacactctcccctgtt

SEQ ID NO: 11 - DNA, 20 bp, Artificial sequence
  CMV forward primer
tggaaatccccgtgagtcaa

SEQ ID NO: 12 - DNA, 19 bp, Artificial sequence
  CMV reverse primer
catggtgatgcggttttgg

SEQ ID NO: 13 - DNA, 22 bp, Artificial sequence
  Synthetic Probe
ccgctatccacgcccattgatg

SEQ ID NO: 14 - DNA, 1431 bp, Homo sapiens
atggaatggagctgggtctttctcttcttcctgtcagtaactacaggtgtccactcccaggttcagctggttcagtccggggctgaggtgaagaagcctggggcctcagtgaaggtttcttgtcaggcttctggatacagattcagtaactttgttattcattgggtgcgccaggcccccggacagaggtttgagtggatgggatggatcaatccttacaacggaaacaaagaattttcagcgaagttccaggacagagtcacctttaccgcggacacatccgcgaacacagcctacatggagttgaggagcctcagatctgcagacacggctgtttattattgtgcgagagtggggccatatagttgggatgattctccccaggacaattattatatggacgtctggggcaaagggaccacggtcatcgtgagctcagcttccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 15 - protein, 476 aa, Homo sapiens
MEWSWVFLFFLSVTTGVHSQVQLVQSGAEVKKPGASVKVSCQASGYRFSNFVIHWVRQAPGQRFEWMGWINPYNGNKEFSAKFQDRVTFTADTSANTAYMELRSLRSADTAVYYCARVGPYSWDDSPQDNYYMDVWGKGTTVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - DNA, 708 bp, Homo sapiens
atgggtgtgcccactcaggtcctggggttgctgctgctgtggcttacagatgccagatgtgagatcgttctcacgcaggctccaggcaccctgtctctgtctccaggggaaagagccaccttctcctgtaggtccagtcacagcattcgcagccgccgcgtacgctggtaccagcacaaacctggccaggctccaaggctggtcatacatggtgtttccaatagggcctctggcatctcagacaggttcagcggcagtgggtctgggacagacttcactctcaccatcaccagagtggagcctgaagactttgcactgtactactgtcaggtctatggtgcctcctcgtacacttttggccaggggaccaaactggagaggaaacgaactgtgcctgcaccatctgtcttcatcttcccgccatctgatgagcagttgaaatctgggactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagatcgcccgtcacaaagagcttcaacaggggagagtgttaa

SEQ ID NO: 17 - protein, 235 aa, Homo sapiens
MGVPTQVLGLLLLWLTDARCEIVLTQAPGTLSLSPGERATFSCRSSHSIRSRRVRWYQHKPGQAPRLVIHGVSNRASGISDRFSGSGSGTDFTLTITRVEPEDFALYYCQVYGASSYTFGQGTKLERKRTVPAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC

SEQ ID NO: 18 - protein, 122 aa, Homo sapiens
LLESGPGLLKPSETLSLTCTVSGGSMINYYWSWIRQPPGERPQWLGHIIYGGTTKYNPSLESRITISRDISKSQFSLRLNSVTAADTAIYYCARVAIGVSGFLNYYYYMDVWGSGTAVTVSS

SEQ ID NO: 19 - protein, 105 aa, Homo sapiens
LTQSPATLSLSPGERATLSCRASQSVGRNLGWYQQKPGQAPRLLIYDASNRATGIPARFSGSGSGTDFTLTISSLEPEDFAVYYCQQRSDWPRTFGQGTKVEIKR

SEQ ID NO: 20 - DNA, 840 bp, Homo sapiens
  CDS (1)..(840)
atgtggtggcgcctgtggtggctgctgctgctgctgctgctgctgtggcccatggtgtgggccgatattgtgctgacgcagtctccaggcaccctgtctttgtctgcaggggaaagagccaccctctcctgcagggccagtcagagtgttagcagcggctccttagcctggtaccagcagaaacctggtcaggctcccaggctcctcatctacggtgcatccaccagggccactggcatcccagacaggttcagtggcagtgggtctgggacagacttcactctcacaatcggcagactggagcctgaagatctcgcagtatattactgtcagcagtatggtacctcaccgtacacttttggccaggggaccaaagtggatatcaaacgtggtggcggtggctcgggcggtggcggttcaggtggcggtggctctagatcttcccaggtccagcttgtgcagtctggggctgaggtgaagaagcctgggtcctcggtgcaggtctcctgcaaggcctctggaggcaccttcagcatgtatggtttcaactgggtgcgacaggcccctggacatggccttgagtggatgggagggatcatccctatctttggtacatcaaactacgcacagaagttccggggcagagtcacgtttaccgcggaccaagccacgagcacagcctacatggagctgaccaacctgcgatctgacgacacggccgtctattattgtgcgagagattttggccccgactgggaagacggtgattcctatgatggtagtggccgggggttctttgacttctggggccagggaaccctggtcaccgtctcctcatga

SEQ ID NO: 21 - protein, 279 aa, Homo sapiens
MWWRLWWLLLLLLLLWPMVWADIVLTQSPGTLSLSAGERATLSCRASQSVSSGSLAWYQQKPGQAPRLLIYGASTRATGIPDRFSGSGSGTDFTLTIGRLEPEDLAVYYCQQYGTSPYTFGQGTKVDIKRGGGGSGGGGSGGGGSRSSQVQLVQSGAEVKKPGSSVQVSCKASGGTFSMYGFNWVRQAPGHGLEWMGGIIPIFGTSNYAQKFRGRVTFTADQATSTAYMELTNLRSDDTAVYYCARDFGPDWEDGDSYDGSGRGFFDFWGQGTLVTVSS

SEQ ID NO: 22 - DNA, 1446 bp, Homo sapiens
  CDS (1)..(1446)
atgtggtggcgcctgtggtggctgctgctgctgctgctgctgctgtggcccatggtgtgggccaggatcacgttaaaggaatcgggtcctccgctggtgaaacccacacagactctcacgctgacctgttccttctctgggttctcactgtccgattttggagtgggtgtaggctggatccgtcagcccccaggaaaggccctagagtggcttgcaatcatttattcggatgatgataagcgctacagcccatcgctgaacaccagactcaccatcaccaaggacacctccaaaaatcaagttgtccttgtcatgactagggtgagtcctgtggacacagccacgtatttctgtgcacacaggaggggccccaccaccctgttcggcgtgcccatcgccaggggccccgtgaacgccatggacgtgtggggccagggcatcaccgtgaccatcagcagcgccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgagctgctgggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccttccccgcccccgagaagaccagcaaggccaagggccagcccagggagccccaggtgtacaccctgccccccagcagggacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgaaccacgaggccctgcacaaccactacacccagaagagcctgagcctgagccccggcaagtag

SEQ ID NO: 23 - protein, 481 aa, Homo sapiens
MWWRLWWLLLLLLLLWPMVWARITLKESGPPLVKPTQTLTLTCSFSGFSLSDFGVGVGWIRQPPGKALEWLAIIYSDDDKRYSPSLNTRLTITKDTSKNQVVLVMTRVSPVDTATYFCAHRRGPTTLFGVPIARGPVNAMDVWGQGITVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPEKTSKAKGQPREPQVYTLPPSRDELTKNQVSLTCLVKGFYPSDIAVEWESNGQPENNYKTTPPVLDSDGSFFLYSKLTVDKSRWQQGNVFSCSVNHEALHNHYTQKSLSLSPGK

SEQ ID NO: 24 - DNA, 714 bp, Homo sapiens
  CDS (1)..(714)
atgcgccccacctgggcctggtggctgttcctggtgctgctgctggccctgtgggcccccgcccgcggcgccctccaactgacccagtctccgtcctccttgtctgcatctgttggagacagaatcaccatcacttgtcgggcaagtcagggcgttaccagtgctttagcctggtatcgacagaagccaggaagtcctcctcaactcctaatctatgatgcctcctctttagaaagtggggtcccatcgaggttcagcggcagtggttctgggacggagttcactctcaccatcagcaccctgcggcctgaagattttgcaacttattactgtcaacaattacatttttaccctcacaccttcggcggcggcaccagggtggacgtgaggcggactgtggctgcaccatctgtcttcatcttcccgccatctgatgagcagttgaaatctgggactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgttaa

SEQ ID NO: 25 - protein, 237 aa, Homo sapiens
MRPTWAWWLFLVLLLALWAPARGALQLTQSPSSLSASVGDRITITCRASQGVTSALAWYRQKPGSPPQLLIYDASSLESGVPSRFSGSGSGTEFTLTISTLRPEDFATYYCQQLHFYPHTFGGGTRVDVRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2012/0027798 A1
(43) Pub. Date: Feb. 2, 2012

(54) ANTIBODY GENE TRANSFER AND RECOMBINANT AAV THEREFOR

(75) Inventors: Kelly Reed Clark, Columbus, OH (US); Philip R. Johnson, JR., Wynnewood, PA (US)

(73) Assignee: NATIONWIDE CHILDREN'S HOSPITAL, INC., COLUMBUS, OH (US)

(21) Appl. No.: 13/177,338

(22) Filed: Jul. 6, 2011

Related U.S. Application Data
(63) Continuation of application No. 12/145,873, filed on Jun. 25, 2008, now abandoned.
Application No. 12/145,873 is a continuation of application No. 10/409,938, filed on Apr. 9, 2003, now abandoned.
(60) Provisional application No. 60/371,501, filed on Apr. 9, 2002.

(51) Int. Cl. A61K 39/21; A61P 37/04; C12P 21/06; C07H 21/04; C12N 5/10

(52) U.S. Cl. 424/208.1; 536/23.53; 435/325; 435/69.6

(57) ABSTRACT

The present invention relates generally to the use of recombinant adeno-associated viruses (rAAV) for gene delivery and more specifically to the use of rAAV to deliver antibody genes to target cells in mammals. Administration of rAAV encoding antibodies that neutralize the HIV-1 virus is exemplified.

This application is a continuation in part of U.S. Provisional Application Ser. No. 60/371,501 filed Apr. 9, 2002.

### FIELD OF INVENTION

The present invention relates generally to the use of recombinant adeno-associated viruses (rAAV) for gene delivery and more specifically to the use of rAAV to deliver antibody genes to target cells in mammals.

### BACKGROUND

Adeno-associated virus (AAV) is a replication-deficient parvovirus, the single-stranded DNA genome of which is about 4.7 kb in length including 145 nucleotide inverted terminal repeat (ITRs). The nucleotide sequence of the AAV serotype 2 (AAV2) genome is presented in Srivastava et al., J. Virol., 45: 555-564 (1983) as corrected by Ruffing et al., J. Gen. Virol., 75: 3385-3392 (1994). Cis-acting sequences directing viral DNA replication (rep), encapsidation/packaging and host cell chromosome integration are contained within the ITRs. Three AAV promoters, p5, p19, and p40 (named for their relative map locations), drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p4-0 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

When wild type AAV infects a human cell, the viral genome can integrate into chromosome 19 resulting in latent infection of the cell. Production of infectious virus does not occur unless the cell is infected with a helper virus (for example, adenovirus or herpesvirus). In the case of adenovirus, genes E1A, E1B, E2A, E4 and VA provide helper functions. Upon infection with a helper virus, the AAV provirus is rescued and amplified, and both AAV and adenovirus are produced.

AAV possesses unique features that make it attractive as a vaccine vector for expressing immunogenic peptides/polypeptides and as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA such as a gene cassette containing a promoter, a DNA of interest and a polyadenylation signal. The rep and cap proteins may be provided in trans. Another significant feature of AAV is that it is an extremely stable and hearty virus. It easily withstands the conditions used to inactivate adenovirus (56° to 65° C. for several hours), making cold preservation of rAAV-vectors less critical. AAV may even be lyophilized. Finally, AAV-infected cells are not resistant to superinfection.

HIV-1 is considered to be the causative agent of Acquired Immunodeficiency Syndrome (AIDS) in the United States. As assessed by the World Health Organization, more than 40 million people are currently infected with HIV and 20 million people have already perished from AIDS. Thus, HIV infection is considered a worldwide pandemic.

There are two currently recognized strains of HIV, HIV-1 and HIV-2. HIV-1 is the principal causes of AIDS around the world. HIV-1 has been classified based on genomic sequence variation into clades. For example, Clade B is the most predominant in North America, Europe, parts of South America and India; Clade C is most predominant in Sub-Saharan Africa; and Clade E is most predominant in southeastern Asia. HIV-1 infection occurs primarily through sexual transmission, transmission from mother to child or exposure to contaminated blood or blood products.

There are two currently recognized strains of HIV, HIV-1 and HIV-2. HIV-1 is the principal causes of AIDS around the world. HIV-1 has been classified based on genomic sequence variation into clades. For example, Clade B is the most predominant in North America, Europe, parts of South America and India; Clade C is most predominant in Sub-Saharan Africa; and Clade E is most predominant in southeastern Asia. HIV-1 infection occurs primarily through sexual transmission, transmission from mother to child or exposure to contaminated blood or blood products.

HIV-1 consists of a lipid envelope surrounding viral structural proteins and an inner core of enzymes and proteins required for viral replication and a genome of two identical linear RNAs. In the lipid envelope, viral glycoprotein 41 (gp 41) anchors another viral envelope glycoprotein 120 (gp 120) that extends from the virus surface and interacts with receptors on the surface of susceptible cells. The HIV-1 genome is approximately 10,000 nucleotides in size and comprises nine genes. It includes three genes common to all retroviruses, the gag, pol and env genes. The gag gene encodes the core structural proteins, the env gene encodes the gp120 and gp41 envelope proteins, and the pol gene encodes the viral enzymes reverse transcriptase (RT), integrase and protease (pro). The genome comprises two other genes essential for viral replication, the tat gene encoding a viral promoter transactivator and the rev gene which also facilitates gene transcription. Finally, the nef, vpu, vpr, and vif genes are unique to lentiviruses and encode polypeptides the functions of which are described in Trono, Cell, 82: 189-192 (1995).

The process by which HIV-1 infects human cells involves interaction of proteins on the surface of the virus with proteins on the surface of the cells. The common understanding is that the first step in HIV infection is the binding of HIV-1 glycoprotein (gp) 120 to cellular CD4 protein. This interaction causes the viral gp120 to undergo a conformational change and bind to other cell surface proteins, such as CCR5 or CXCR4 proteins, allowing subsequent fusion of the virus with the cell. CD4 has thus been described as the primary receptor for HIV-1 while the other cell surface proteins are described as coreceptors for HIV-1.

HIV-1 infection is characterized by an asymptomatic period between infection with the virus and the development of AIDS. The rate of progression to AIDS varies among infected individuals. AIDS develops as CD4-positive cells, such as helper T cells and monocytes/macrophages, are infected and depleted. AIDS is manifested as opportunistic infections, increased risk of malignancies and other conditions typical of defects in cell-mediated immunity. The Centers for Disease Control and Prevention clinical categories of pediatric, adolescent and adult disease are set out in Table I of Sleasman and Goodenow, J. Allergy Clin. Immunol., 111(2): S582-S592 (2003).

Predicting the likelihood of progression to AIDS involves monitoring viral loads (viral replication) and measuring CD4-positive T cells in infected individuals. The higher the viral loads, the more likely a person is to develop AIDS. The lower the CD4-positive T cell count, the more likely a person a person is to develop AIDS.

At present, antiretroviral drug therapy (ART) is the only means of treating HIV infection or preventing HIV-1 transmission from one person to another. At best, even with ART, HIV-1 infection is a chronic condition that requires lifelong drug therapy and there can still be a slow progression to disease. ART does not eradicate HIV-1 because the virus can persist in latent reservoirs. Moreover, treatment regimens can be toxic and multiple drugs must be used daily. There thus is an urgent need to develop effective vaccines and treatments for HIV-1 infection.

Over the past several years, progress has been made towards a safe and effective vaccine for HIV infection, and multiple approaches have been taken in animal models and humans. Many of the vaccine candidates have elicited measurable and significant antigen-specific T cell responses. In contrast, many fail to induce serum antibodies that broadly neutralize primary isolates of HIV-1. Thus, if one considers such antibodies to be an important defense against HIV-1 infection and disease, there remains a significant gap in the design of current HIV-1 vaccine candidates.

There are several hypotheses put forth to explain the lack of neutralizing antibody induction after vaccination with envelope immunogens. First, most anti-envelope antibodies elicited do not recognize the mature oligomeric envelope complex, but rather bind to unprocessed gp160 precursor or monomeric gp120. This is due in part to the trimeric structure of the mature envelope spike, which yields a molecule of low inherent immunogenicity. Extensive glycosylation of surface exposed domains renders a significant portion of the spike non-immunogenic, giving rise to the so-called “silent face” of the molecule. Second, the compact structure of the trimeric moiety sterically interferes with antibody recognition of protein epitopes that are located within the core of the trimer. Importantly, these same epitopes are readily exposed on the unprocessed gp160 precursor or monomeric gp120 proteins and map to the non-neutralizing face of the protein. Consequently, it has been extremely difficult to isolate human monoclonal antibodies that neutralize primary viral isolates in a broad, cross-clade manner. In fact, as few as six such antibodies (b12, 2G12, 2F5, Z13, and 4E10 described in Zwick et al., J. Virol., 75: 12198-12208, 2001 and X5 described in Moulard et al., Proc. Natl. Acad. Sci. USA, 6913-6918, 2002) have been identified despite efforts using a variety of techniques. The fact that such antibodies are rare in HIV-1 infected humans serves to underscore the ill-defined but substantial obstacles in eliciting broadly reactive antibodies using traditional methods of vaccination.

One potential solution to this problem might be to prophylactically administer antibody preparations (monoclonal or polyclonal) that possess the desired neutralizing activities. With regard to HIV-1, studies in non-human primates suggest that passively administered neutralizing antibodies can provide significant protection against SIV/SHIV/HIV infection. This type of “passive immunization” scheme has been successfully applied on a large scale to a targeted population of infants at risk for serious respiratory syncytial virus infection. However, such a strategy for HIV infection has significant drawbacks. It would be cost prohibitive and impractical to frequently administer antibody preparations to large numbers of people for an indefinite period of time.

### SUMMARY OF INVENTION

The present invention recognizes the need for development of effective preventative and therapeutic treatments for HIV-1 infection. Because of the significant obstacles associated with both active and passive immunization strategies, the present invention utilizes an approach which exploits the existence of the aforementioned neutralizing human monoclonal antibodies against HIV-1 gp160 and the unique gene-delivery properties of AAV.

In a first aspect, the invention provides rAAV genomes. The rAAV genomes comprise AAV ITRs flanking a gene cassette of DNA encoding one or more antibody polypeptides operatively linked to transcriptional control DNA, specifically promoter DNA and polyadenylation signal sequence DNA, functional in target cells. The gene cassette may also include intron sequences to facilitate processing of the RNA transcript when expressed in mammalian cells. The rAAV genomes of the invention lack AAV rep and cap DNA. AAV DNA in the rAAV genomes may be from any AAV serotype for which a recombinant virus can be derived.

In particular, the invention contemplates a dual promoter gene cassette which encodes light and heavy chain polypeptides. In one embodiment, the gene cassette contains the following: (1) two constitutive promoters that are active in the cell that will be transduced, (2) several unique restriction enzyme sites to allow for the rapid replacement of promotor elements or heavy and light chain coding sequences, (3) unique restriction sites that facilitate in-frame antibody gene cloning, (4) a strong transcriptional termination site 3′ to the first expression cassette to reduce possible promoter interference and (5) polynucleotide sequences encoding both the heavy and light chain of a monoclonal antibody of interest each inserted under the transcriptional control of one of the two promoters.

The invention contemplates rAAV genomes which express antibodies directed to viral proteins (for example, proteins of HIV, Hepatitis B virus, Hepatitis C virus, Epstein Barr Virus and Respiratory Syncytial Virus) and bacterial proteins as well as other proteins associated with chronic disease states (such as a protein expressed only when the disease state occurs or a protein whose expression is upregulated compared to expression in the absence of the disease state) where provision of antibodies would be an effective treatment. Examples of chronic disease states include cancers, inflammatory diseases such as rheumatoid arthritis and inflammatory bowel diseases, and prion associated diseases such as Mad Cow Disease, Creutzfeldt-Jakob disease, Gerstmann-Straussler-Scheinker syndrome, fatal familial insomnia, kuru, and Alpers syndrome. Genomes encoding monoclonal antibodies which neutralize primary HIV-1 isolates such as monoclonal antibodies IgG1b12, 2F5, Z13, 45E10, F105 and X5 are specifically contemplated. These antibodies are variously described in Zwick et al., J. Virol., 75: 12198-12208, 2001; Baba et al., Nat. Med. 6: 200-206, 2000; and Moulard et al., Proc. Natl. Acad. Sci. USA, 6913-6918, 2002). Amino acid sequences of Z13 are set out in Genebank Accession Nos. AY035845 and AY035846 (SEQ ID NOs: 18 and 19) and heavy and light chain DNA and amino acid sequences of 2F5 are respectively in SEQ ID NOS: 22 AND 23 and SEQ ID NOS: 24 and 25. Specifically exemplified herein is a dual promoter gene cassette comprising the human CMV immediate early promoter/enhancer, the SV40 small T-antigen intron, b12 heavy chain coding sequences, the bovine growth hormone polyadenylation site, the human elongation factor-1α promoter modified using the R segment and part of the U5 sequence of the HTLV Type 1 Long Terminal Repeat, the 1117 intron, b12 light chain coding sequences and the SV40 polyadenylation site. A rAAV genome comprising that gene cassette is designated the rAAV/IgG1b12 genome. Also specifically exemplified herein is a gene cassette comprising the human CMV immediate early promoter/enhancer, the SV40 small T-antigen intron, X5 heavy chain variable region coding sequences, a (Gly₃Ser)₄ linker, X5 light chain variable region coding sequences and the SV40 polyadenylation site. A rAAV genome comprising that gene cassette is designated the rAAV/ScFvX5 genome.

The invention contemplates antibody polypeptides encoded by the rAAV genomes may be intact immunoglobulin molecules of any class (IgG, IgA, IgD, IgM or IgE) or subclasses thereof, tetramers, dimers, single chain antibodies, bifunctional antibodies, chimeric antibodies, humanized antibodies, wholly novel recombinant antibodies, antibodies synthesized de novo by chemical or biological means, Fab fragments, F(ab)₂′, and other immunoactive portions, fragments, segments and other smaller or larger partial antibody structures, wherein all possess sufficient binding activity so as to be therapeutically useful within the methods of the present invention. In addition, since the invention contemplates the use of “humanized” antibodies, for example, some variation in the identity of the framework and/or complementarity determining regions (i.e., the CDRs or hypervariable regions of the heavy and light chain variable regions of said antibodies that are critical to determining the antigenic specificity of the antibodies) is both permitted and expected. Thus, in accordance with the present invention, the amino acid sequence(s) of the antibody polypeptides useful in the present invention may show some sequence variation from those antibodies of known utility in treating infections (acute and chronic infections) and chronic diseases. Such sequence variations may be as much as 5%, thereby an antibody sequence useful herein has at least a 95% identity with an antibody of known utility. As noted above, antibody polypeptides may be (or be derived from) any antibody class. Antibody class may be selected (or modified) by those skilled in the art taking into account the infection and/or disease state being treated and the desired action(s) and site(s) of action of the antibody polypeptides.

In another aspect, the invention provides DNA vectors comprising rAAV genomes of the invention. The vectors are transferred to cells permissible for infection with a helper virus of AAV (e.g., adenovirus, E1-deleted adenovirus or herpesvirus) for assembly of the rAAV genome into infectious viral particles. Techniques to produce rAAV particles, in which a AAV genome to be packaged, rep and cap genes, and helper virus functions are provided to a cell are standard in the art. Production of rAAV requires that the following components are present within a single cell (denoted herein as a packaging cell): a rAAV genome, AAV rep and cap genes separate from the rAAV genome, and helper virus functions. The AAV rep and cap genes may be from any AAV serotype for which recombinant virus can be derived and may be from a different AAV serotype than the rAAV genome ITRs.

A method of generating a packaging cell is to create a cell line that stably expresses all the necessary components for AAV particle production. For example, a plasmid (or multiple plasmids) comprising a rAAV genome, AAV rep and cap genes separate from the rAAV genome, and a selectable marker, such as a neomycin resistance gene, are integrated into the genome of a cell. The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

The invention thus provides packaging cells that produce infectious rAAV. In one embodiment packaging cells may be stably transformed cancer cells such as HeLa cells, 293 cells and PerC.6 cells (a cognate 293 line). In another embodiment, packaging cells are cells that are not transformed cancer cells such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

In another aspect, the invention provides rAAV (i.e., infectious encapsidated rAAV particles) comprising a rAAV genome of the invention. In one embodiment, the rAAV is rAAV/IgG1b12. In another embodiment the rAAV is rAAV/ScFvX5. The rAAV may be purified by methods standard in the art such as by column chromatography or cesium chloride gradients.

In another embodiment, the invention contemplates compositions comprising rAAV of the present invention. These compositions may be used to treat and/or prevent viral infections (acute and chronic viral infections) in particular AIDS, bacterial infections (acute, subacute and chronic bacterial infections) and other chronic disease states. In one embodiment, compositions of the invention comprise a rAAV encoding an antibody polypeptide of interest. In other embodiments, compositions of the present invention may include two or more rAAV encoding different antibody polypeptides of interest. In particular for neutralizing HIV-1, administration of a rAAV mixture which results in expression and secretion of several anti-HIV-1 antibody polypeptides may increase neutralization of the virus. Administration may precede, accompany or follow ART.

Compositions of the invention comprise rAAV in a pharmaceutically acceptable carrier. The compositions may also comprise other ingredients such as diluents and adjuvants. Acceptable carriers, diluents and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycol (PEG).

Titers of rAAV to be administered in methods of the invention will vary depending, for example, on the particular rAAV, the mode of administration, the treatment goal, the individual, and the cell type(s) being targeted, and may be determined by methods standard in the art.

Methods of transducing a target cell with rAAV, in vivo or in vitro, are contemplated by the invention. The in vivo methods comprise the step of administering an effective dose or doses of a composition comprising a rAAV of the invention to an animal (including a human being) in need thereof. If the dose is administered prior to infection by a virus or development of a chronic disease state, the administration is prophylatic. If the dose is administered after infection by a virus or development of a chronic disease state, the administration is therapeutic. An effective dose is a dose sufficient to alleviate (eliminate or reduce) at least one symptom associated with the infection or disease state being treated. In one embodiment, alleviation of symptoms prevents progression of a viral infection to a disease state. In another embodiment, alleviation of symptoms prevents progression to, or progression of, a disease state (whether or not caused by a viral infection). Viral infections (including acute and chronic viral infections), bacterial infections (including acute, subacute and chronic infections) and chronic diseases states of a patient to be treated include, but are not limited to, HIV-1 infection, Hepatitis B virus infection, Hepatitis C virus infection, Epstein Barr Virus infection, Respiratory Syncytial Virus infection, osteomyelitis, tuberculosis, rheumatoid arthritis, inflammatory bowel disease, transmissible spongiform encephalopathies such as Creutzfeldt-Jakob disease and kuru, Gerstmann-Straussler-Scheinker syndrome, fatal familial insomnia, Alpers syndrome, and cancers. In the methods, rAAV encoding antibody polypeptides specifically reactive with a proteins or peptide associated with the infection or disease state are administered. Administration of an effective dose of the compositions may be by routes standard in the art, for example, parenteral, intravenous, oral, buccal, nasal, pulmonary, intracranial, intraosseous, intraocular, rectal, or vaginal. Route(s) of administration and serotype(s) of AAV components of rAAV (in particular, the AAV ITRs and capsid protein) of the invention may be chosen and/or matched by those skilled in the art taking into account the infection and/or disease state being treated and the target cells/tissue(s) that are to express the antibody polypeptides.

In particular, actual administration of rAAV of the present invention may be accomplished by using any physical method that will transport the rAAV recombinant vector into the target tissue of an animal. Simply resuspending a rAAV in phosphate buffered saline has been demonstrated to be sufficient to provide a vehicle useful for muscle tissue expression, and there are no known restrictions on the carriers or other components that can be coadministered with the vector (although compositions that degrade DNA should be avoided in the normal manner with vectors). Capsid proteins of a rAAV may be modified so that the rAAV is targeted to a particular target tissue of interest such as muscle. Pharmaceutical compositions can be prepared as injectable formulations or as topical formulations to be delivered to the muscles by transdermal transport. Numerous formulations for both intramuscular injection and transdermal transport have been previously developed and can be used in the practice of the invention. The rAAV can be used with any pharmaceutically acceptable carrier for ease of administration and handling.

For purposes of intramuscular injection, solutions in an adjuvant such as sesame or peanut oil or in aqueous propylene glycol can be employed, as well as sterile aqueous solutions. Such aqueous solutions can be buffered, if desired, and the liquid diluent first rendered isotonic with saline or glucose. Solutions of rAAV as a free acid (DNA contains acidic phosphate groups) or a pharmacologically acceptable salt can be prepared in water suitably mixed with a surfactant such as hydroxpropylcellulose. A dispersion of rAAV can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonger absorption of the injectable compositions can be brought about by use of agents delaying absorption, for example, aluminum monostearate and gelatin.

Sterile injectable solutions are prepared by incorporating rAAV in the required amount in the appropriate solvent with various of the other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique which yield a powder of the active ingredient plus any additional desired ingredient from the previously sterile-filtered solution thereof.

Transduction with rAAV can also be carried out in vitro. In one embodiment, desired target muscle cells are removed from the subject, transduced with rAAV and reintroduced into the subject. Alternatively, syngeneic or xenogeneic muscle cells can be used where those cells will not generate an inappropriate immune response in the subject.

Suitable methods for the transduction and reintroduction of transduced cells into a subject are known in the art. In one embodiment, cells can be transduced in vitro by combining rAAV with muscle cells, e.g., in appropriate media, and screening for those cells harboring the DNA of interest using conventional techniques such as Southern blots and/or PCR, or by using selectable markers. Transduced cells can then be formulated into pharmaceutical compositions, and the composition introduced into the subject by various techniques, such as by intramuscular, intravenous, subcutaneous and intraperitoneal injection, or by injection into smooth and cardiac muscle, using e.g., a catheter.

Transduction of cells with rAAV of the invention results in sustained expression of antibody polypeptide(s). The present invention thus provides methods of delivering rAAV which express antibody polypeptides to an animal, preferably a human being. These methods include transducing tissues (including but not limited to muscle, liver and brain) with one or more rAAV of the present invention. Transduction may be carried out with gene cassettes comprising tissue specific control elements. For example, one embodiment of the invention provides methods of transducing muscle cells and muscle tissues directed by muscle specific control elements, including, but not limited to, those derived from the actin and myosin gene families, such as from the myoD gene family (See: Weintraub et al., Science 251: 761-766, 1991), the myocyte-specific enhancer binding factor MEF-2 (Cserjesi and Olson, Mol. Cell. Biol. 11: 4854-4862, 1991), control elements derived from the human skeletal actin gene (Muscat et al., Mol. Cell. Biol. 7: 4089-4099, 1987), the cardiac actin gene, muscle creatine kinase sequence elements (See: Johnson et al. Mol. Cell. Biol. 9:3393-3399, 1989) and the murine creatine kinase enhancer (mCK) element, control elements derived from the skeletal fast-twitch troponin C gene, the slow-twitch cardiac troponin C gene and the slow-twitch troponin I gene: hypozia-inducible nuclear factors (Semenza et al., Proc. Natl. Acad. Sci. USA 88: 5680-5684, 1991), steroid-inducible elements and promoters including the glucocorticoid response element (GRE) (See: Mader and White, Proc. Natl. Acad. Sci. USA 90: 5603-5607, 1993), and other control elements.

Muscle tissue is a attractive target for in vivo gene delivery and gene therapy, because it is not a vital organ and is easy to access. To carry out the delivery of the rAAV in the methods of the present invention to muscle cells and tissue, the use of single-chain antibody (scFv) or Fab derivatives is contemplated to facilitate more efficient antibody secretion from the muscle tissue. One single chain antibody contemplated by the invention is the HIV-1 neutralizing single chain antibody X5, the DNA and amino acid sequences of which are not out in SEW ID NOs: 20 and 21, respectively. In addition, rAAV based on alternate serotypes (e.g. AAV-1 and AAV-5) may transduce skeletal myocytes more efficiently than AAV-2.

rAAV have been shown to transduce muscle cells or muscle tissue with high efficiency and direct the long-term expression of a variety of transgenes. Because of the flexibility of this system, it is contemplated that light and heavy chain antibody genes can be incorporated into a single rAAV, and the antibody-expressing rAAV can then be used to transduce muscle in vivo. The invention contemplates sustained expression of biologically active antibody polypeptides from transduced myofibers.

By “muscle cell” or “muscle tissue” is meant a cell or group of cells derived from muscle of any kind, including skeletal muscle, smooth muscle, e.g. from the digestive tract, urinary bladder and blood vessels, cardiac, and excised from any area of the body. Such muscle cells may be differentiated or undifferentiated, such as myoblasts, myocytes, myotubes, cardiomyocytes and cardiomyoblast. Since muscle tissue is readily accessible to the circulatory system, a protein produced and secreted by muscle cells and tissue in vivo will logically enter the bloodstream for systemic delivery, thereby providing sustained, therapeutic levels of protein secretion from muscle.

The term “transduction” is used to refer to the delivery of antibody polypeptide DNA to a recipient cell either in vivo or in vitro, via a replication-deficient rAAV of the invention resulting in expression of a functional antibody polypeptide by the recipient cell.

It is known that antibodies directed to HIV-1 can neutralize the virus by prohibiting viral binding to its receptor(s). The invention provides methods of administering an effective dose (or doses) of rAAV of the present invention that encode antibody polypeptide(s) that neutralize a virus to a patient in need thereof. Neutralization according to the invention is a reduction in infectivity of a primary viral isolate as measured by an in vitro or in vivo assay known in the art. Multiple assays are known in the art. Neutralization may involve one or more of the following binding of antibody to antigen on the surface of the virus blocking interaction of the virus with a receptor on a cell, binding of antibody to antigen on the surface of the virus resulting in complement-mediated lysis and/or phagocytosis of the virus, binding of antibody to antigen on the surface of cells infected with the virus resulting in activation of Fc-mediated effector systems and lysis/clearance of the infected cell by antibody-dependent cellular cytotoxicity (ADCC) or complement-dependent cytotoxicity, binding of antibody to antigen on the surface of cells infected with the virus resulting in inhibition of viral replication, binding of antibody to antigen on the surface of cells infected with the virus resulting in inhibition of virus release from the infected cells, and binding of antibody to antigen on the surface of cells resulting in inhibition of cell-cell transmission of the virus. Neutralization by intracellular immunization is also contemplated. Furthermore, neutralization of bacteria is contemplated.

Neutralization may result in clearance of a virus or bacteria from the patient (i.e., sterilization) or may slow progression to a disease state caused by a virus or bacteria. In one embodiment, methods of the invention include the administration of an effective dose (or doses) of rAAV of the invention encoding HIV-1 neutralizing antibody polypeptides to prevent progression of a patient infected with HIV-1 to AIDS. Preferred methods result in one or more of the following in the individual: a reduction of viral loads, maintenance of low viral loads, an increase in CD4-positive T cells, stabilization of CD4-positive T cells, reduced incidence or severity of opportunistic infections, reduced incidence of malignancies, and reduced incidence or severity of conditions typical of defects in cell-mediated immunity. The foregoing are each in comparison to an individual that, according to the art, has progressed or will likely progress to AIDS.

As described herein (see Example 5), significant levels of HIV-1 neutralizing activity are found in the sera of mice for over six months after a single intramuscular administration of a rAAV vector of the present invention which expresses the anti-HIV-1 monoclonal antibody IgG1b12. This approach allows for predetermination of antibody affinity and specificity prior to “immunization”, and avoids the need for an active humoral immune response against the HIV envelope protein.

### BRIEF DESCRIPTION OF DRAWING

FIG. 1 depicts a dual promoter rAAV genome designated pCMV/HC/EF1a/LC. Unique restriction sites are labeled at the top of the schematic. Genome components are labeled as follows: “HC” and “LC” denote the heavy and light chain antibody genes, respectively, “CMVp” represents the human CMV immediate early promoter/enhancer, and “I” denotes the SV40 small T-antigen intron. Antibody leader sequences are labeled “L”, and “pA” denotes the bovine growth hormone polyadenylation site. The second transcriptional unit contains the human elongation factor-1α (EF-1α) promoter, and has been modified to enhance stability of DNA and RNA using the R segment and part of the U5 sequence (R-U5′) of the HTLV Type 1 Long Terminal Repeat. This promoter also contains the 1117 intron, which is derived from plasmid pGT62LacZ (InVivoGen Inc.). The light chain polyadenylation site is from SV40.

FIG. 2 depicts the rAAV/X5 genome that contains a gene cassette comprising the human CMV immediate early promoter/enhancer (denoted “CMV”), the SV40 small T-antigen intron (“SV40 sd/sa”), X5 heavy chain variable region coding sequences (“V_{H}”), a (Gly₃Ser)₄ linker (“linker”), X5 light chain variable region coding sequences (“V_{L}”) and the SV40 polyadenylation site (“SV40 poly A”).

### DETAILED DESCRIPTION

The following examples illustrate the invention wherein Example 1 describes construction of a dual promoter rAAV for antibody polypeptide expression, Example 2 describes rAAV production, Example 3 describes production of circulating IgG₁ in rAAV transduced mice, Example 4 describes neutralization of HIV-1 activity by muscle-derived IgG1b12 and Example 5 describes rAAV persistence and IgG1b12 production in muscle. Example 6 describes construction of a single promoter rAAV for antibody polypeptide expression Example 7 describes rAAV administration to macaques and Example 8 to humans. Example 9 describes rAAV useful in the invention including rAAV that encode antibody polypeptides other than those useful for viral infections.

### Example 1

### Construction of a Dual Promoter rAAV for Antibody Expression

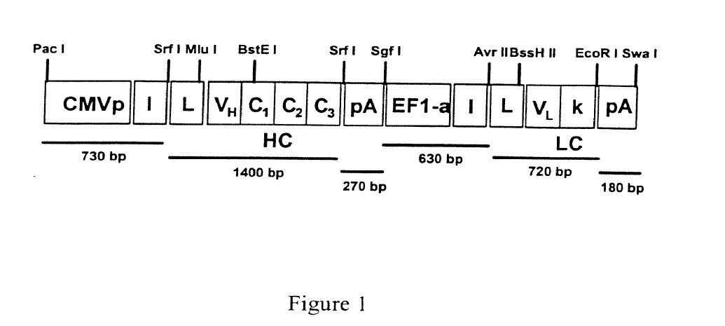

To achieve efficient antibody expression within target muscle cells, a dual promoter rAAV was constructed that resulted in optimal co-expression of heavy and light chain proteins within the same transduced cell. As shown in FIG. 1, the resulting dual promoter rAAV had the following features: (1) two constitutive promoters that are active in skeletal muscle in the context of a rAAV vector (hCMV promoter/enhancer and the human EF1-alpha promoter); (2) several unique 8 basepair restriction enzyme sites incorporated into the vector to allow for the rapid replacement of promotor elements or heavy and light chain coding sequences; (3) site-directed mutagenesis was performed on the heavy and light chain leader peptide sequences of IgG1b12 to introduce unique restriction sites (Mlu I for the heavy chain leader and BssH II for the light chain leader) that facilitate in-frame antibody gene cloning; (4) the IgG1b12 heavy chain introns were removed by RT-PCR to reduce vector size and remain within the packaging limit of wild-type AAV; and (5) a strong transcriptional termination site 3′ to the first expression cassette to reduce possible promoter interference. Lastly, to enable high-titer rAAV/IgG1b12 vector production using a stable producer cell line approach (Clark et al., Hum. Gene Ther. 6:1329-1341, 1995), the rAAV/IgG1b12 plasmid vector sequences were cloned into a larger tripartite plasmid (pAAV/IgG1b12/rep-cap/neotk) that also contains the AAV-2 rep-cap helper sequences and a neomycin resistance gene as previously described (Clark et al., Hum. Gene Ther. 6:1329-1341, 1995). The tripartite plasmid was then used to generate an optimal HeLa based, rAAV/IgG1b12 producer cell line (CE71).

The coding sequences for the light and heavy chains of the human monoclonal antibody, IgG1b12, were derived from plasmid pDR12 and are set out at SEQ ID NOS: 14 and 16, respectively herein. The resulting amino acid sequences for the heavy and light chains of IgG1b12 are set out as SEQ ID NOS: 15 and 17, respectively herein. Isolation of IgG1b12 and plasmid pDR12 have been described in Burton et al., Proc. Natl. Acad. Sci. USA 88: 10134-10137, 1991 and Science 266: 1024-1027 (1994).

The dual promoter rAAV cloning plasmid pAAV/IgG1b12 was constructed sequentially as follows. First, plasmid pCMV/β (Clontech) was digested with Pst I and the 2.7 kb vector plasmid DNA fragment isolated and re-ligated with itself to generate a ampicillin resistant vector (pB). PCR was then used to amplify the hCMV promoter/enhancer and SV40 intron (808 bp) from plasmid pCMV/β and was carried out with the following primers: CMV forward: TCTAGAATTCTTTAATTAAGTCGTTA CATAACTTACGG (SEQ ID NO: 1); CMV reverse: TCTAGAATTCTGCCCGGG CTACAATTCCGCAGCTTTTAG (SEQ ID NO: 2).

The resulting fragment was cloned into the unique EcoR I site of plasmid pB to generate plasmid pCMV. These primers were designed with EcoR I sites flanking unique Pac I and Srf I sites at the 5′ and 3′ ends. Subsequently, PCR was used to amplify the bovine growth hormone polyadenylation signal (190 bp) and the EF1-α promoter (770 bp) separately using plasmid pGT621acZ as a template (InVivoGen) and the following primers respectively: BGH forward: TTAGTGTGCCCGGGCACTCGCTGATCAGCCTCGACT (SEQ ID NO: 3); BGH reverse: TAGTGTCTCGAGAATCCTCCCCCTTGCTGTC (SEQ ID NO: 4); EF1 forward: TTAGTGTCTCGAGAACTAACATACGCTCTCCA (SEQ ID NO: 5); EF1 reverse: GTGTCTGCAGGTATTTAAATGTGGGAATTCGTCCTAGGCCCTC CTACCGGTGATCTC (SEQ ID NO: 6). The resulting PCR fragments were subsequently Xho I digested, re-ligated, and a second round of PCR performed with the BGH forward and EF1-α reverse primers (SEQ ID NOS: 3 and 6) to generate a single DNA fragment. These primers incorporated a 5′ Srf I site and 3′ Avr II, EcoR I, Swa I, and Pst I sites, respectively. This 960 bp DNA fragment was directionally cloned into plasmid pCMV at the unique Srf and Pst I sites. The resulting plasmid (pCMV/EF1) now possessed the CMV promoter with a BGH polyadenyation site followed by the EF1-alpha promoter.

A 270 base pair DNA fragment containing the SV40 polyadenylation signal (isolated from plasmid pGT621acZ) was directionally cloned into the EcoR I/Swa I sites of plasmid pCMV/EF1 to yield pCMV/EF1a. The IgG1b12 heavy chain cDNA (1,463 bp) was isolated by transfecting CHO cells with plasmid pDR12 and isolating total RNA. The RNA was subjected to RT-PCR and cloned into the pZero vector (Invitrogen) with the following primers: heavy chain cDNA forward: TACTTCGCCCGGGCTAATTCGCCGCC ACCATGGAA (SEQ ID NO: 8); heavy chain cDNA reverse: TACTTCGCC CGGGCTTTATTCATTTACCCGGAGACAGGG (SEQ ID NO: 9). These primers incorporated flanking Srf I sites that facilitated the cloning of the IgG1b12 heavy chain into the unique Srf I site in plasmid pCMV/EF1a, yielding plasmid pCMV/HC/EF1a. The IgG1b12κ light chain gene was PCR amplified directly from plasmid pDR12 and the 720 bp product cloned into plasmid pZero with the following primers: light chain forward: CCTCACCTAGGCCACCATGGGTGTGCCACGCTGG (SEQ ID NO: 9); light chain reverse: CCTCACCTAGGATTAACACTCTCCCCTGTT (SEQ ID NO: 10). The light chain primers incorporated flanking Avr II restriction sites that were used to clone the kappa light chain into the Avr II site of plasmid pCMV/HC/EF1a to generate plasmid pCMV/HC/EF1a/LC.

Site directed mutagenesis was then used to introduce a unique Mlu I restriction site into the heavy chain leader peptide sequence and a similar strategy was employed to introduce a BssH I site into the light chain leader peptide sequence. The dual expression cassette was then isolated as a 4.5 kb Pac I/Srf I DNA fragment and cloned between the AAV ITRs of plasmid pAAV/β-gal/rep-cap/neotk (Clark et al., Hum. Gene Therapy 6: 1329-1341, 1995) to generate pAAV/IgG1b12/rep-cap/neotk. This tripartite plasmid contains the native rep-cap AAV helper sequences, as well as, the neomycin resistance gene for stable cell line selection.

The ability of the plasmid pAAV/IgG1b12/rep-cap/neotk and rAAV/IgG1b12 to produce human IgG1 antibody was initially confirmed in vitro using several transformed cell lines (CHO-K1, HeLa, COS-7 and C2C12). Following plasmid transfection or rAAV/IgG1b12 transduction using standard methods in the art, cell culture supernatant was analyzed using the 1 human IgG subtype 1 ELISA Immunoassay kit (The Binding Site) as directed by the manufacturer. The sensitivity of this assay is 2.9 mg/ml of human IgG. This assay determined that the cell culture supernatant contained detectable levels of human IgG₁.

### Example 2

### rAAV Production

rAAV/IgG1b12 was produced and purified using methods known in the art (Clark et al., Hum. Gene Therapy 10: 1031-1039, 1999; Clark et al., Hum. Gene Therapy 6: 1329-1341, 1995). Briefly, a producer cell line (CE71) was isolated following HeLa cell transfection with plasmid pAAV/IgG1b12/rep-cap/neotk and subsequent G418 (700 μg/drug selection. Two hundred individual cell lines were screened following wild-type adenovirus type 5 infection (moi=20) and CE71 was identified as producing the highest DNase resistant particles (DRP) per cell (10⁴ DRP/cell). For large scale vector production, 10¹⁰ CE71 cells were expanded in a Corning Cell Cube adherent cell bioreactor and subsequently infected with wild-type Ad 5 (moi=20). Following development of adenovirus CPE (72 hr), rAAV/IgG1b12 was purified from the crude CE71 cell lysate using heparin chromatography as previously detailed (Clark et al., Hum. Gene Therapy 10: 1031-1039, 1999). DRP titers were determined for purified rAAV/IgG1b12 by real time PCR methodology utilizing a Prism 7700 Taqman sequence detector system (PE Applied Biosystems) as detailed in Clark et al., 1999. The primer and fluorescent probe set used for rAAV/IgG1b12 quantitation were as follows; CMV forward primer: 5′-TGGAAATCCCCGTGAGTCAA-3′ (SEQ ID NO: 11), CMV reverse primer: 5′-CATGGTGATGCGGTTTTGG-3 (SEQ ID NO: 12), and probe, 5-FAM-CCGCTATCCACGCCCATTGATG-TAMRA-3′ (SEQ ID NO: 13).

An infectious rAAV/IgG1b12 titer was determined using serial dilutions of the rAAV/IgG1b12 stock and infecting a rep-cap expressing cell line (C12) in the presence of adenovirus. An end point titer determination was made based on quantitative PCR detection of replicating rAAV/IgG1b12 genomes in C12 cells, as previously described (Clark et al., Gene Therapy 3: 1124-1132, 1996). The calculated DRP to IU ratio of rAAV/IgG1b12 used in these experiments was 28:1.

### Example 3

### Production of Circulating IgG₁ in rAAV Transduced Mice

Immunodeficient Rag1 mice were inoculated with rAAV/IgG1b12 into both quadriceps muscles. Rag1 mice were used to avoid an anti-human IgG response.

All experiments were conducted in accordance with the Children's Hospital Institutional Animal Care and Use Committee. Six week old Rag-1 mice (C.129S7(B6)-Rag 1^{tmIMom}) were purchased from The Jackson Laboratory (Bar Harbor, Me.) and housed in microisolator barrier housing. The study consisted of 16 animals: 6 received 5×10¹¹ DNase resistant particles (DRP) of rAAV/IgG1b12; 6 received 5×10¹⁰ DRP; 2 received an irrelevant rAAV vector expressing β-glucuronidase (rAAV/GUS, 4×10¹¹ DRP); and, 2 were given PBS diluent (used for vector DNA analysis only).

Mice were anesthetized with intramuscular injection of tiletamine HCl/zolezapam HCl (Telazol, Ft. Dodge, Iowa). A 5 mm skin incision was made over the distal femur and 50 μl of the viral suspension or PBS was injected in the quadriceps femoris muscle along the long axis of the muscle using a 28-gauge needle. No adverse effects attributable to the injection procedure were noted in any mice. Blood samples were collected from the retroorbital-sinus under anesthesia. At the time of sacrifice, the entire quadriceps femoris muscles were removed and bisected along the transverse plane and half fixed in a non-cross-linking fixative (Histochoice, Amresco, Solon, Ohio) and paraffin embedded, and the other half quick frozen in liquid nitrogen for subsequent DNA analysis. Tibialis anterior muscle was taken as control tissue.

Human IgG₁ was detected at 6 weeks post-inoculation in all eleven surviving rAAV/IgG1b12 mice; one mouse died at 2 weeks from unrelated causes. On average, animals that received the higher dose of rAAV/IgG1b12 possessed 7 to 28 times more IgG₁ than lower dose animals. Maximal IgG1 concentrations were observed 12 weeks after injection, and then plateaued over the next 3 months. The majority of high dose animal sera consistently possessed between 4 and 5 μg of human IgG1 per ml, with the maximum level exceeding 8 μg/ml. In the low dose group, antibody levels continued to increase 20 weeks after vector inoculation with circulating antibody levels in the 0.5-1.0 μg/ml range.

Several sera were also assayed to determine an anti-HIV gp120 end-point ELISA titer. The anti-HIV-1 gp120 ELISA was carried out as follows. Immulon 4 immunoassay plates (Dynatech) were coated (100 ng/well) with recombinant HIV-1_{LAI} gp120 produced in Chinese hamster ovary (CHO) cells (Quality Biological, Gaithersburg, Md.) diluted in carbonate buffer (BupH, Pierce, Rockford, Ill.) for 16 hours at 4° C. Antigen was removed and the wells were blocked with 1% normal goat serum in Blotto (5% skim dry milk in 1×PBS pH 7.4) for 1 hour at 25° C. Mouse sera were diluted in 0.1% (v/v) Triton-X100 in PBS and incubated for 30 minutes, then washed 5 times by immersion in 0.1% (v/v) Triton-X100 in PBS. A goat anti-human IgG1 HRP conjugated secondary antibody (1:5,000) was added for 1 hr (Pierce, Rockford, Ill.). The colorometric substrate, 3,3′,5,5′ tetramethylbenzidine (TMB) was added and the reaction stopped after 30 minutes with the addition of 1 N H₂SO₄. Both ELISA assays were read at 450 nm on a Perkin-Elmer HTS 7000 plate reader. Endpoint titers were derived by taking the reciprocal of the serum dilution that yielded OD₄₅₀ values that were at least 2 times higher than the corresponding no antigen control wells.

Serial dilutions serum taken at 16 weeks from 4 higher dose animals revealed endpoint titers in the range of 1:800-1:3,200. These data confirmed that muscle secreted IgG1b12 retained gp120 binding specificity.

### Example 4

### Muscle-derived IgG1b12 Neutralizes HIV-1

While the IgG₁ and gp120 ELISA data confirmed in vivo antibody expression, these assays did not address whether secreted IgG1b12 retained the ability to neutralize HIV-1. Therefore, 20 week serum samples from the 6 high dose animals were analyzed for neutralization activity against TCLA strain HIV-1 IIIB using the MT-2 cell-killing assays. These assays utilized Finter's neutral red to quantify viable cells as described in Herzog et al., Proc. Natl. Acad. Sci. USA 94: 5804-5809, 1997. Titers were reported ac the reciprocal serum dilution at which 50% of cells were protected from virus-induced killing. This 50% end-point corresponds to >90% reduction in p24 Gag antigen synthesis in this assay (See Bures et al., AIDS Res. Hum. Retroviruses 16: 2019-2035, 2000). Neutralization of SHIV-89.6 was measured in mitogen-stimulated human peripheral blood mononuclear cells (PBMC) by using a reduction in p27 Gag antigen synthesis as described in Bures et al., supra. Virus stocks were generated in either H9 cells (HIV-1 IIIB) or human PBMC (SHIV-89.6).

Sera from 5 of the 6 animals possessed detectable neutralization activity as described below in Table 1.

TABLE 1
Serum neutralization titers against HIV-1 IIIB.

| | | | Predicted | Observed human |
|---|---|---|---|---|
| | Weeks after injection | | IgG1b12 | IgG₁ |
| | (titer)^{b} | | concentration | concentration |
| Mouse^{a} | 0 | 20 | (μg/ml)^{c} | (μg/ml)^{d} |
| RA1 | <20 | 93 | 6.7 | 5.1 |
| RA2 | <20 | 56 | 4.0 | 5.9 |
| RA3 | <20 | 55 | 4.0 | 6.0 |
| RB1 | <20 | 44 | 3.2 | 5.3 |
| RB2 | <20 | 47 | 3.4 | 4.0 |
| RB3 | <20 | <20 | 0.0 | 2.8 |

^{a}Each mouse received a single dose of 5 × 10¹¹ DRP of rAAV/IgG1b12 in the quadriceps.
^{b}Antibody-mediated neutralization was measured in an MT-2 cell-killing assay. Titers are the reciprocal serum dilution at which 50% of cells were protected from virus-induced killing, which corresponds to >90% reduction in p24 Gag antigen synthesis.
^{c}Concentration of purified IgG1b12 that is necessary to achieve the observed neutralization titer.
^{d}Actual concentration of human IgG1 in sera.

To extend these data, the ability of mouse sera to neutralize a primary-like HIV-1 isolate (SHIV-89.6 containing the HIV 89.6 env) was also tested. Neutralization activity was observed in serum pools from 16, 20 and 24 weeks after injection (Table 2); sera were pooled because of limited volumes. These data indicated that IgG1b12 originating from muscle retained the predicted ability to neutralize both TCLA and primary HIV-1 isolates.

TABLE 2
Serum neutralization titers against SHIV 89.6.

| Test sample^{a} | | p27 (pg/ml) | % reduction^{b} |
|---|---|---|---|
| Diluent | | 1725 | 0 |
| IgG1b12 (4 μg/ml) | | 89 | 95 |
| Pooled | 0 | 711 | 59 |
| mouse sera | 16 | 225 | 87 |
| (week after | 20 | 326 | 81 |
| injection) | 24 | 527 | 69 |

^{a}Pre-immune (time 0) pool consisted of sera from mice RA1, RA2, RA3, RBI, RB2, and RB3. Sera from these same mice were pooled by collection date (16, 20, or 24 wks after injection) as indicated. All pools were assayed at a 1:4 dilution.
^{b}Percent reduction in p27 antigen was calculated relative to the amount of p27 synthesized in the absence of serum.

### Example 5

### rAAV Vector Persistence and Protein Production in Muscle

To obtain evidence that muscle was the site of antibody production, rAAV genome persistence and human IgG protein expression was assayed in muscle tissue harvested 24 weeks post-inoculation. rAAV/IgG1b12 vector DNA persistence was analyzed using real-time quantitative PCR. Approximately 50% of the left and right quadriceps from each animal were used for genomic DNA isolation and subjected to Taqman PCR using a PCR primer/probe pair specific for the CMV promoter. As shown in Table 3, all inoculated muscle tissue possessed significant levels of vector DNA that ranged between 0.4-10 copies per nucleus. On average, muscle from animals that received the higher dose possessed 5 times more vector DNA per muscle nucleus than muscle from low dose animals.

TABLE 3
Persistence of vector DNA in mouse muscle.

| Vector/dose^{a} | Mouse | Genome copies/nucleus^{b} |
|---|---|---|
| PBS | RF3 | 0.003 |
| | RF4 | 0.012 |
| rAAV/IgG1b12 | RC1 | 9.0 |
| 5 × 10¹⁰ | RC2 | 5.5 |
| | RC3 | 0.5 |
| | RD2 | 1.2 |
| | RD3 | 4.5 |
| rAAV/IgG1b12 | RA1 | 42.5 |
| 5 × 10¹¹ | RA2 | 3.2 |
| | RA3 | 20.0 |
| | RB1 | 27.1 |
| | RB2 | 0.4 |
| | RB3 | 19.3 |

^{a}Dose is measured as DNase resistant particles (DRP); see Materials and Methods.
^{b}Values represent the average rAAV genomes per nucleus observed in the quadriceps muscles following rAAV injection. 60 ng of muscle DNA (10,000 nuclei equivalents) was analyzed by quantitative Taqman PCR using the CMV primer/probe set (SEQ ID NO: 1 AND 2). All samples were harvested 24 weeks after injection.

To demonstrate in situ antibody expression within the inoculated muscle, immunoperoxidase staining for human IgG1 was performed on paraffin embedded muscle tissue. Muscle tissue for human IgG₁ heavy chain (Fc specific) detection was serial sectioned (6 μm) and deparaffinized in Americlear with successive ethanol baths followed by a 1×PBS+0.2% Tween 20 wash. Tissue sections were initially processed using the Antigen Retrieval Citra Solution (BioGenex) according to the manufacturer's instructions and then blocked for 10 minutes using Power Block reagent (SioGenex). A 1:100 dilution of a polyclonal rabbit anti-human IgG antiserum (DAKO, A0424) was incubated with the sections for 18 hr at 4° C. After extensive washing, a biotinylated anti-rabbit secondary antibody (1:100 dilution; Vector Laboratories) was added and incubated for 30 minutes. Antigen was visualized using an avidin/biotin-peroxidase conjugate according to the manufacturer's instructions (VECTASTAIN Elite ABC-peroxidase, Vector Laboratories). Color development was achieved by incubating the sections for 5 minutes in AEC peroxidase substrate (DAKO).

All higher dose animals demonstrated appreciable, often punctate, immunostaining of specific myofibers consistent with ER/golgi localization of the secreted protein. Control rAAV/GUS quadriceps tissue was negative for the presence of human IgG1 (animals RF1, RF2).

### Example 6

### Construction of a Single Promoter rAAV for Antibody Expression

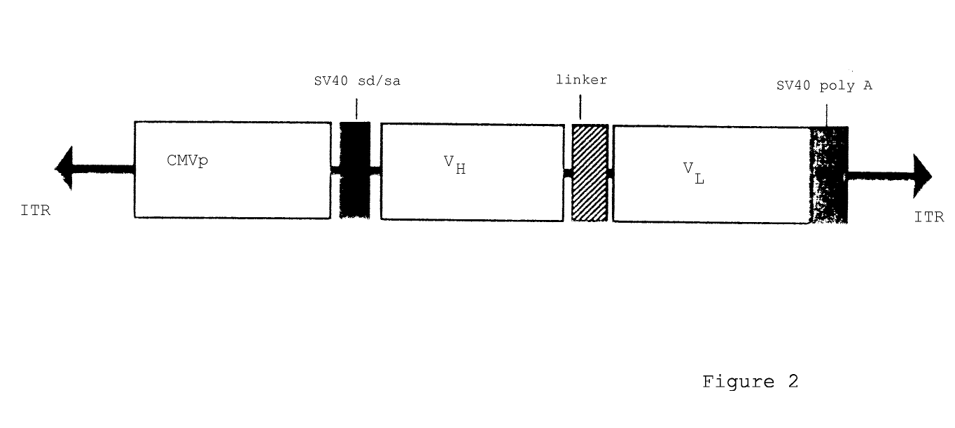

A rAAV genome comprising AAV ITRs flanking a gene cassette comprising the human CMV immediate early promoter/enhancer, the SV40 small T-antigen intron, X5 heavy chain variable region coding sequences, a (Gly₃Ser)₄ linker, X5 light chain variable region coding sequences and the SV40 polyadenylation site was constructed using standard DNA manipulation techniques. The gene cassette encoded a single chain X5 antibody polypeptide (ScFvX5). See FIG. 2. The AAV ITRs were modified as described in McCarty et al., Gene Therapy, 8, 124801254, 2001 to allow for packaging of the rAAV genome into viral particles as double-stranded, self-complimentary DNA.

The ability of the gene cassette to produce ScFvX5 was confirmed in vitro using HeLa cells. The gene cassette was transiently transfected into HeLa cells as naked DNA. The ScFvX5 produced by the transfected cells and was demonstrated to bind HIV-1, gp120 by standard methods in the art.

The rAAV/ScFvX5 genome was packaged essentially by the methods described in Example 2 above but was packaged into AAV serotype 1 capsid and then purified. The resulting rAAV/ScFvX5 may be tested in the mouse model described in Examples 3, 4 and 5 above and the macaque model described in Example 6 below.

### Example 7

### RAAV Administration to Macaques

Infection of macaque monkeys with SIV can induce AIDS-like disease. These infected animals can be employed as a model for infection of humans with HIV-1 and development of AIDS in humans. In particular, macaques infected with SIV virus develop manifestations characteristic of human AIDS such as depletion of CD4-positive T cells, development of opportunistic infections, neurological diseases and malignancies, and the like. Moreover, SIV can infect animals by routes of administration (e.g., rectal and vaginal) that reproduce the transmission of HIV in humans. See, for example, Nathanson, International Journal of STD & AIDS, 9(Suppl. 1):3-7 (1998). The SIV/macaque model is thus the leading animal model for AIDS vaccine development and parthenogenesis. See, for example, Ho et al., Cell, 110:135-138 (2002) (discussing that the success of strategies employed in monkey experiments has “propelled a number of candidate vaccines into clinical trials”). See also, grantsl.nih.gov/grants/guide/rfa-files/RFA-MH-99-009.html (Jan. 29, 1999) (discussing the “importance of the SIV model of AIDS to aid in deciphering and understanding the mechanisms underlying human AIDS neuropathogenesis”).

Moreover, challenge studies in monkeys can be employed to establish protection against immunodeficiency viruses and/or the diseases resulting from infection with the viruses and are tools for establishing whether a vaccine or vaccine concept holds promise for its use in humans. See Schultz, AIDS Research and Human Retroviruses, 14(3):S261-S263 (1998).

The macaque model can thus be utilized to confirm the beneficial effects of administration of rAAV/IgG1b12 and/or rAAV encoding other HIV-1 neutralizing antibody. For example, rAAV/IgGlb12 is administered by one or more intramuscular injection(s) to macaques prior, or subsequent to, infection with a challenge HIV-1 virus such as SIVsm/E660 (a viral strain that induces a more AIDS-like disease in macaques presenting a more stringent challenge than other commonly-employed challenge viruses). Neutralizing antibody levels are measured at various time points as described in Polacino et al., J. Virol., 73(10): 8201-8215 (1999).

Administration of the rAAV results in one or more of the following in macaques exposed to SIV: a reduction of viral loads, maintenance of low viral loads, an increase in CD4-positive T cells, stabilization of CD4-positive T cells, reduced incidence or severity of opportunistic infections, reduced incidence of malignancies, and reduced incidence or severity of conditions typical of defects in cell-mediated immunity. The foregoing are each in comparison to a macaque that, according to the art, has progressed or will likely progress to AIDS-like disease.

### Example 8

### Administration of rAAV Encoding HIV-1 Antibody Polypeptides to Humans

rAAV/IgG1b12, rAAV/ScFvX5, rAAV encoding other HIV-1 antibody polypeptide (or a mixture of two or more of the following) is administered to an individual susceptible to infection by HIV-1 or infected by HIV-1 to prevent or slow progression to AIDS.

For example, one or more intramuscular injection(s) of rAAV is(are) administered to the individual. Levels of production of antibody polypeptide encoded by the rAAV are monitored by techniques standard in the art. Likelihood of progression to AIDS is monitored by measurement, by techniques standard in the art, of HIV-1 viral loads and CD4-positive Tcells. The higher the viral loads, the more likely the individual is to develop AIDS. The lower the CD4-positive T cell count, the more likely the individual is to develop AIDS.

Administration of the rAAV results in one or more of the following in the individual when infected with HIV-1: a reduction of viral loads, maintenance of low viral loads, an increase in CD4-positive T cells, stabilization of CD4-positive T cells, reduced incidence or severity of opportunistic infections, reduced incidence of malignancies, and reduced incidence or severity of conditions typical of defects in cell-mediated immunity. The foregoing are each in comparison to an individual that, according to the art, has progressed or will likely progress to AIDS.

### Example 9

### rAAV Encoding Other Antibody Polypeptides

Other rAAV may be generated and used according to the methods described herein such as rAAV encoding antibody polypeptides Orthoclone OKT3 for allograft rejection, ReoPro as a PTCA adjunct, Rituxan for non-Hodgkin's lymphoma, Simulect for organ rejection, Remicade for rheumatoid arthritis and Crohn's disease, Zenapax for organ rejection, Synagis for RSV infection, Herceptin for metastatic breast cancer, Mylotarg for acute myeloid leukemia, Campath for chronic lymphocytic leukemia, Zevalin for non-Hodgkin's lymphoma and Humira for rheumatoid arthritis. These have been approved for human use for the infections or disease states noted.

While the present invention has been described in terms of specific embodiments, it is understood that variations and modifications will occur to those skilled in the art. Accordingly, only such limitations as appear in the claims should be placed on the invention.

1. The rAAV/IgG1b12 genome.

2. A polynucleotide comprising the rAAV/IgG1b12 genome of claim 1.

3. A packaging cell comprising the rAAV/IgG1b12 genome of claim 1.

4. A purified rAAV/IgG1b12 comprising the rAAV/IgG1b12 genome of claim 1.

5. A composition comprising the rAAV/IgG1b12 of claim 4.

6. A method of producing IgG1b12 antibody in a cell comprising the step of transducing a cell with the purified rAAV/IgG1b12 of claim 4 in an amount effective to elicit expression of IgG1b12 antibody in said cell.

7. A method of producing IgG1b12 antibody in an animal comprising the step of administering to said patient the composition of claim 5 in an amount effective to elicit expression of IgG1b12 antibody in said animal.

8. The method of claim 7 wherein said animal is a human being.

9. The rAAV/ScFvX5 genome.

10. A polynucleotide comprising the rAAV/ScFvX5 genome of claim 9.

11. A packaging cell comprising the rAAV/ScFvX5 genome of claim 9.

12. A purified rAAV/ScFvX5 comprising the rAAV/ScFvX5 genome of claim 9.

13. A composition comprising the rAAV/ScFvX5 of claim 12.

14. A method of producing ScFvX5 antibody polypeptide in a cell comprising the step of transducing a cell with the purified rAAV/ScFvX5 of claim 12 in an amount effective to elicit expression of ScFvX5 antibody polypeptide in said cell.

15. A method of producing ScFvX5 antibody polypeptide in an animal comprising the step of administering to said patient the composition of claim 13 in an amount effective to elicit expression of ScFvX5 antibody polypeptide in said animal.

16. The method of claim 15 wherein said animal is a human being.

17. A purified rAAV encoding an anti-HIV-1 antibody polypeptide.

18. A composition comprising one or more rAAV of claim 17.

19. A method of producing anti-HIV-1 antibody polypeptide in a cell comprising the step of transducing a cell with the rAAV of claim 17 in an amount effective to elicit expression the anti-HIV-1 antibody polypeptide in said cell.

20. A method of producing anti-HIV-1 antibody polypeptide in an animal comprising the step of administering to said patient the composition of claim 19 in an amount effective to elicit expression of the anti-HIV-1 antibody polypeptide in said animal.

21. The method of claim 20 wherein said animal is a human being.
